(19) United States
(12) Patent Application Publication
(10) Pub. No.: US 2026/0114991 A1
(43) Pub. Date: Apr. 30, 2026

(54) TRANSCATHETER HEART VALVE PROSTHESIS WITH LEAFLET PROTECTION FEATURES

(71) Applicant: MEDTRONIC, INC., Minneapolis, MN (US)

(72) Inventors: Matthew C. Baldwin, Santa Rosa, CA (US); Thuy Linh L. Dinh, Westminster, CA (US)

(73) Assignee: MEDTRONIC, INC., Minneapolis, MN (US)

(21) Appl. No.: 19/368,886

(22) Filed: Oct. 24, 2025

Related U.S. Application Data
(60) Provisional application No. 63/712,391, filed on Oct. 25, 2024.

(51) Int. Cl. A61F 2/24

(52) U.S. Cl. CPC A61F 2/2418; A61F 2230/0004; A61F 2250/0037; A61F 2250/0039

(57) ABSTRACT

A transcatheter heart valve prosthesis includes a frame including an inflow portion, an outflow portion, and a transition portion, and a prosthetic valve coupled to the frame. The outflow portion includes a plurality of endmost outflow crowns and a plurality of outflow struts with each endmost outflow crown being formed between a pair of opposing outflow struts. Each endmost outflow crown has an inner edge and an outer edge. When the transcatheter valve prosthesis is in an as-cut configuration, a first center point of a first radius of curvature of the inner edge is offset from a second center point of a second radius of curvature of the outer edge such that the endmost outflow crowns flare radially outward when the frame is radially expanded.

### CROSS-REFERENCE TO RELATED APPLICATIONS

This application claims the benefit of U.S. Provisional Application No. 63/712,391, filed Oct. 25, 2024, the contents of which are incorporated herein in their entirety.

### FIELD OF THE INVENTION

The present invention relates to transcatheter valve prostheses that are radially expandable by a balloon.

### BACKGROUND OF THE INVENTION

A human heart includes four heart valves that determine the pathway of blood flow through the heart: the mitral valve, the tricuspid valve, the aortic valve, and the pulmonary valve. The mitral and tricuspid valves are atrioventricular valves, which are between the atria and the ventricles, while the aortic and pulmonary valves are semilunar valves, which are in the arteries leaving the heart. Ideally, native leaflets of a heart valve move apart from each other when the valve is in an open position and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Flexible prosthetic valves supported by a stent structure (a frame) that can be delivered percutaneously using a catheter-based delivery system have been developed for heart valve replacement. These transcatheter heart valve prostheses may include either self-expanding or balloon-expandable stent structures with valve leaflets attached to the interior of the stent structure. Such transcatheter heart valve prostheses can be reduced in diameter, by crimping onto a balloon catheter or by being contained within a sheath component of a delivery catheter, and advanced through the venous or arterial vasculature. Once the heart valve prosthesis is positioned at the treatment site, for instance within an incompetent native valve, the stent structure may be expanded to hold the prosthetic valve firmly in place.

In some cases, when a transcatheter heart valve prosthesis is implanted in a native heart valve, such as a native valve with an extreme elliptical annulus, the valve leaflets of the transcatheter prosthetic valve may contact the frame of the transcatheter heart valve prosthesis, possibly causing damage to the prosthetic valve leaflets and reducing durability of the transcatheter heart valve prosthesis. Valve leaflets may also be pinched between frame components during crimping of the heart valve prosthesis. Such pinching may damage the prosthetic valve leaflets and/or reduce durability of the transcatheter heart valve prosthesis. Embodiments hereof relate to an improved transcatheter valve prosthesis configured to minimize leaflet contact with the frame and reduce the risk of leaflet pinching during crimping.

### BRIEF SUMMARY OF THE INVENTION

The details of one or more aspects of the disclosure are set forth in the accompanying drawings and the description below. Other features, objects, and advantages of the techniques described in this disclosure will be apparent from the description and drawings, and from the claims

In an example hereof, the present disclosure relates to a transcatheter valve prosthesis. The transcatheter valve prosthesis includes: a frame formed in an as-cut configuration, the frame being radially expandable to a radially expanded configuration and radially compressible to a crimped configuration for delivery within a vasculature; and a prosthetic valve coupled to the frame. The frame includes: an inflow portion, an outflow portion, and a transition portion. The inflow portion is formed proximate to an inflow end of the frame, the inflow portion including a plurality or rows of inflow struts and inflow crowns, with each inflow crown being formed between a pair of opposing inflow struts. The outflow portion is formed proximate to an outflow end of the frame, the outflow portion including a plurality of endmost outflow crowns and a plurality of outflow struts with each endmost outflow crown being formed between a pair of opposing outflow struts. The transition portion extends between the inflow portion and the outflow portion, the transition portion including a plurality of axial frame members, the plurality of axial frame members including a plurality of commissure posts. The prosthetic valve is disposed within and secured to at least the transition portion of the frame, the prosthetic valve being configured to substantially block blood flow in one direction to regulate blood flow through a central lumen of the frame. When the frame is in the radially expanded configuration, the outflow end of the frame flares radially outwardly such that the outflow end has a first diameter larger than a second diameter at the inflow end of the frame. Each endmost outflow crown has an inner edge and an outer edge. When the transcatheter valve prosthesis is in the as-cut configuration, a first center point of a first radius of curvature of the inner edge is offset from a second center point of a second radius of curvature of the outer edge such that the endmost outflow crown flares radially outward when the frame is radially expanded.

In another example, in the transcatheter valve prosthesis according to any of the preceding or following examples, the outflow end of the transcatheter valve prosthesis in the as-cut configuration has a third diameter and the inflow end of the frame has a fourth diameter that are about equal.

In another example, in the transcatheter valve prosthesis according to any of the preceding or following examples, the first center point of the first radius of curvature is proximal to the second center point of the second radius of curvature.

In another example, in the transcatheter valve prosthesis according to any of the preceding or following examples, an offset distance between the first center point and the second center point is in the range of about 0.2 mm to about 0.7 mm.

In another example, in the transcatheter valve prosthesis according to any of the preceding or following examples, the first radius of curvature is about 0.45 mm.

In another example, in the transcatheter valve prosthesis according to any of the preceding or following examples, the second radius of curvature is in the range of 1.1 mm.

In another example, in the transcatheter valve prosthesis according to any of the preceding or following examples, each outflow strut of the outflow portion has an s-shape with the frame in the as-cut configuration, and each strut of the outflow portion has a generally linear shape with the frame in the crimped configuration.

In another example, in the transcatheter valve prosthesis according to any of the preceding or following examples, each outflow strut includes a first segment extending from a proximal end of the outflow strut in a distal direction to a second segment extending from the first segment in a distal direction to a corresponding endmost outflow crown.

In another example, in the transcatheter valve prosthesis according to any of the preceding or following examples, the first segment of each outflow strut has a convex shape relative to a centerline of an adjacent axial member of the plurality of axial members with the frame in the as-cut configuration.

In another example, in the transcatheter valve prosthesis according to any of the preceding or following examples, the second segment of each outflow strut has a convex shape relative to the centerline of the adjacent axial member with the frame in the as-cut configuration.

In another example, in the transcatheter valve prosthesis according to any of the preceding or following examples, each commissure post includes an outflow portion such that outflow struts adjacent to each commissure post intersect the commissure post proximal of a free end of the commissure post. When the transcatheter valve prosthesis is in the as-cut configuration, a first circumferential distance is defined between the first segment of a first outflow strut of the plurality of the outflow struts adjacent the commissure post and the commissure post, when the transcatheter valve prosthesis is in the crimped configuration and a second circumferential distance is defined between the first segment of the first outflow strut and the commissure post, wherein the second circumferential distance is larger than the first circumferential distance.

In another example hereof, a transcatheter valve prosthesis includes: a frame and a prosthetic valve. The frame is formed in an as-cut configuration, being radially expandable to a radially expanded configuration and radially compressible to a crimped configuration for delivery within a vasculature and includes: an inflow portion formed proximate to an inflow end of the frame, the inflow portion including a plurality or rows of inflow struts and inflow crowns, with each inflow crown being formed between a pair of opposing inflow struts, an outflow portion formed proximate to an outflow end of the frame, the outflow portion including a plurality of outflow crowns and a plurality of outflow struts with each outflow crown being formed between a pair of opposing outflow struts, and a transition portion extending between the inflow portion and the outflow portion, the transition portion including a plurality of axial frame members, the plurality of axial frame members including a plurality of commissure posts. The prosthetic valve is disposed within and secured to at least the transition portion of the frame with the prosthetic valve being configured to substantially block blood flow in one direction to regulate blood flow through a central lumen of the frame. Each outflow strut of the outflow portion has an s-shape with the frame in the as-cut configuration, and each strut of the outflow portion has a generally linear shape with the frame in the crimped configuration.

In another example, in the transcatheter valve prosthesis according to any of the preceding or following examples, each outflow strut includes a first segment extending from a proximal end of the outflow strut in a distal direction to a second segment extending from the first segment in a distal direction to a corresponding endmost outflow crown.

In another example, in the transcatheter valve prosthesis according to any of the preceding or following examples, the first segment of each outflow strut has a convex shape relative to a centerline of an adjacent axial member of the plurality of axial members with the frame in the as-cut configuration.

In another example, in the transcatheter valve prosthesis according to any of the preceding or following examples, the second segment of each outflow strut has a convex shape relative to the centerline of the adjacent axial member with the frame in the as-cut configuration.

In another example, in the transcatheter valve prosthesis according to any of the preceding or following examples, each commissure post includes an outflow portion such that outflow struts adjacent to each commissure post intersect the commissure post proximal of a free end of the commissure post. When the transcatheter valve prosthesis is in the as-cut configuration, a first circumferential distance is defined between the first segment of a first outflow strut of the plurality of the outflow struts adjacent the commissure post and the commissure post and when the transcatheter valve prosthesis is in the crimped configuration, a second circumferential distance is defined between the first segment of the first outflow strut and the commissure post, wherein the second circumferential distance is larger than the first circumferential distance.

In another example, in the transcatheter valve prosthesis according to any of the preceding or following examples, the outflow portion includes a plurality of endmost outflow crowns formed at the outflow end of the frame and each endmost outflow crown has an inner edge and an outer edge. When the transcatheter valve prosthesis is in the as-cut configuration, a first center point of a first radius of curvature of the inner edge is offset from a second center point of a second radius of curvature of the outer edge such that the endmost outflow crown flares radially outward when the frame is radially expanded.

In another example, in the transcatheter valve prosthesis according to any of the preceding or following examples, the first center point of the first radius of curvature is proximal to the second center point of the second radius of curvature.

In another example, in the transcatheter valve prosthesis according to any of the preceding or following examples, the first radius of curvature is about 0.45 mm.

In another example, in the transcatheter valve prosthesis according to any of the preceding or following examples, the second radius of curvature is about 1.1 mm.

In another example, in the transcatheter valve prosthesis according to any of the preceding or following examples, free distal inflow crowns of the inflow crowns at a distal end of the inflow portion of the frame flare radially outwardly with the transcatheter heart valve prosthesis in the radially expanded configuration.

In another example, in the transcatheter valve prosthesis according to any of the preceding or following examples, each of the free distal inflow crowns has an inner edge and an outer edge, and wherein in the as-cut configuration a third center point of a third radius of curvature of the inner edge is offset from a fourth center point of a fourth radius of curvature of the outer edge such that the free distal inflow crown flares radially outward when the frame is radially expanded.

In another example, in the transcatheter valve prosthesis according to any of the preceding or following examples, the fourth center point is offset from the third center point by about 0.2 mm to about 0.5 mm.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 depicts a perspective view of a transcatheter heart valve prosthesis in a radially expanded configuration according to an embodiment hereof.

FIG. 2 depicts an outflow or top view of the transcatheter heart valve prosthesis of FIG. 1.

FIG. 3 depicts a side view of a frame of the transcatheter heart valve prosthesis according to an embodiment hereof.

FIG. 4 depicts a laid-open view of the frame of FIG. 3 in an as-cut configuration according to embodiments hereof.

FIG. 5 depicts an enlarged view of a portion A of an outflow end of the frame of FIG. 4.

FIG. 6 depicts an enlarged view of a portion B of the outflow end of the frame of FIG. 4.

FIG. 7 depicts a side view of the frame of the FIG. 4 in a non-expanded or crimped configuration.

FIG. 8 depicts an enlarged view of a portion C of the outflow end of the frame of FIG. 7.

FIG. 9 depicts a perspective view of a transcatheter heart valve prosthesis in a radially expanded configuration according to an embodiment hereof.

FIG. 10 depicts an outflow or top view of the transcatheter heart valve prosthesis of FIG. 9.

FIG. 11 depicts a side view of a frame of the transcatheter heart valve prosthesis of FIG. 9 according to an embodiment hereof.

FIG. 12 depicts a laid-open view of the frame of FIG. 11 in an as-cut configuration according to embodiments hereof.

FIG. 13 depicts an enlarged view of a portion of FIG. 12.

FIG. 14A depicts an enlarged view of a portion D of the frame shown in FIG. 13.

FIG. 14B depicts an enlarged view of a portion D of the frame shown in FIG. 13.

FIG. 15 depicts a laid-open view of an as-cut configuration of a frame of another heart valve prosthesis.

FIG. 16 depicts a close-up view of a portion of the frame of FIG. 15.

### DETAILED DESCRIPTION OF THE INVENTION

Specific embodiments of the present disclosure are now described with reference to the figures wherein like reference numbers indicate identical or functionally similar elements. The following detailed description describes examples of embodiments of the invention and is not intended to limit the present technology or the application and uses of the present technology. Although the description of embodiments hereof is in the context of an aortic heart valve prosthesis, the present technology may also be used in other valve prostheses. For example, embodiments transcatheter heart valve prosthesis described herein may be configured for placement within a pulmonary, aortic, mitral, or tricuspid valve, or may be utilized with a valve prosthesis configured for placement within a venous valve or within other body passageways where it is deemed useful. Furthermore, there is no intention to be bound by any expressed or implied theory presented in the preceding Field, Background, Brief Summary, or the following Detailed Description.

The terms “distal” and “proximal,” when used in the following description to refer to a native vessel, native valve, or a device to be implanted into a native vessel or native valve, such as a heart valve prosthesis, are with reference to the direction of blood flow. Thus, “distal” and “distally” refer to positions in a downstream direction with respect to the direction of blood flow, and the terms “proximal” and “proximally” refer to positions in an upstream direction with respect to the direction of blood flow.

Embodiments hereof relate to transcatheter heart valve prostheses with a frame configured to improve valve leaflet durability of the transcatheter heart valve prosthesis. The frame may be configured to prevent valve leaflet contact with the frame when the transcatheter heart valve prosthesis is deployed, for example, and not by way of limitation, in a native valve annulus with an extreme elliptical shape. The frame may be further configured to prevent pinching of the valve leaflets between frame components as the transcatheter heart valve prosthesis is crimped or radially compressed to a crimped or non-expanded configuration or state, for example when loading the transcatheter heart valve prosthesis onto or into a delivery device, for example, crimping a balloon expandable the heart valve prosthesis onto an expandable balloon of a delivery device.

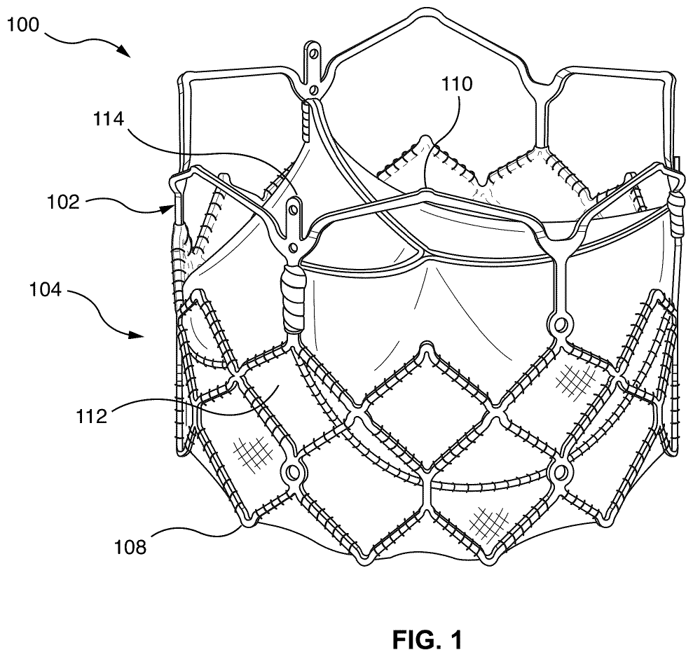

FIGS. 1-8 illustrate a transcatheter heart valve prosthesis 100 according to embodiments hereof. One skilled in the art will realize that FIGS. 1-8 illustrate one example of a transcatheter heart valve prosthesis and that existing components illustrated in FIGS. 1-8 may be removed and/or additional components may be added to the transcatheter heart valve prosthesis 100.

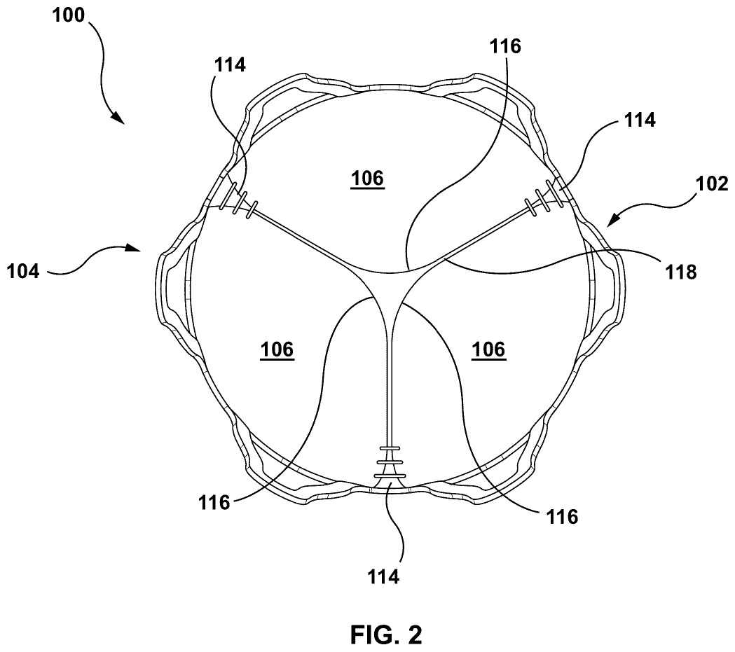

In embodiments herein, the transcatheter heart valve prosthesis 100 includes a radially-expandable frame 102 and a prosthetic valve component 104. The frame 102 is generally tubular and is configured to be in a crimped configuration for delivery within a vasculature and an expanded configuration for deployment within a native heart valve. The frame 102 further includes an as-cut configuration when formed. The prosthetic valve component 104 includes one or more valve leaflets 106. The prosthetic valve component 104 is configured to block flow in one direction to regulate flow there-through via the valve leaflets 106. FIG. 1 is a perspective view of the transcatheter heart valve prosthesis 100 in an expanded configuration. FIG. 2 is an end view of the transcatheter heart valve prosthesis 100. When the transcatheter heart valve prosthesis 100 is deployed within the valve annulus of a native heart valve, the frame 102 of the transcatheter heart valve prosthesis 100 is configured to be radially expanded within native valve leaflets of the patient's defective valve. In embodiments herein, the transcatheter heart valve prosthesis 100 is configured for replacement for an aortic valve such that an inflow end 108 of the transcatheter heart valve prosthesis 100 extends into and anchors within the aortic annulus of a patient's left ventricle, while an outflow end 110 of the transcatheter heart valve prosthesis 100 is positioned within the aortic sinuses.

The prosthetic valve component 104 is disposed within and secured to a portion of the frame 102. As shown in FIG. 2, in the embodiment shown, the prosthetic valve component 104 includes three (3) valve leaflets 106 configured to block flow in one direction to regulate flow there-through. While FIG. 2 illustrates the prosthetic valve component 104 with three leaflets 106, this is not meant to be limiting and the prosthetic valve component 104 may be configured with a single valve leaflet, two valve leaflets, three valve leaflets, four valve leaflets, or more than four valve leaflets. In embodiments herein, the valve leaflets 106 may be attached to a graft material 112 which encloses or lines a portion of the frame 102, as known to one of ordinary skill in the art. The valve leaflets 106 are sutured or otherwise securely and sealingly attached along their bases to the interior surface of the graft material 112, and/or otherwise attached to the frame 102. Adjoining pairs of leaflets 106 are attached to one another at their lateral ends to form commissures 114, with free edges 116 of the valve leaflets 106 forming coaptation edges that meet in area of coaptation 118. The valve leaflets 106 may be made of various natural or synthetic materials, non-limiting examples including heart valves, aortic roots, aortic walls, aortic leaflets, pericardial tissue, such as pericardial patches, bypass grafts, blood vessels, intestinal submucosal tissue, umbilical tissue DACRON® polyester, other cloth materials, nylon blends, polymeric materials, and vacuum deposition nitinol fabricated materials.

The graft material 112 may enclose or line the frame 102 as known to one of ordinary skill in the art of prosthetic tissue valve construction. The graft material 112 may be a natural or biological material such as pericardium or another membranous tissue such as intestinal submucosa, or synthetic materials such as low-porosity woven fabric, such as polyester, Dacron fabric, or PTFE.

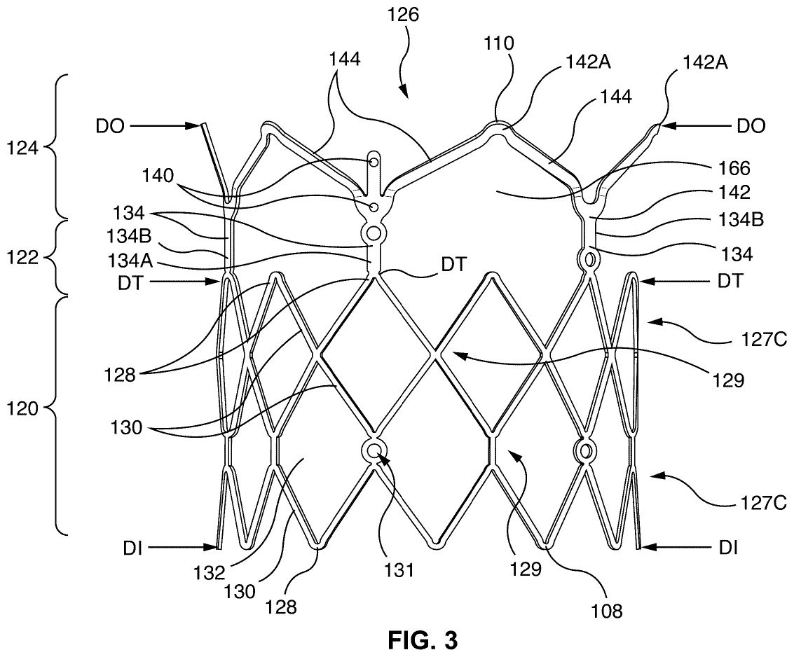

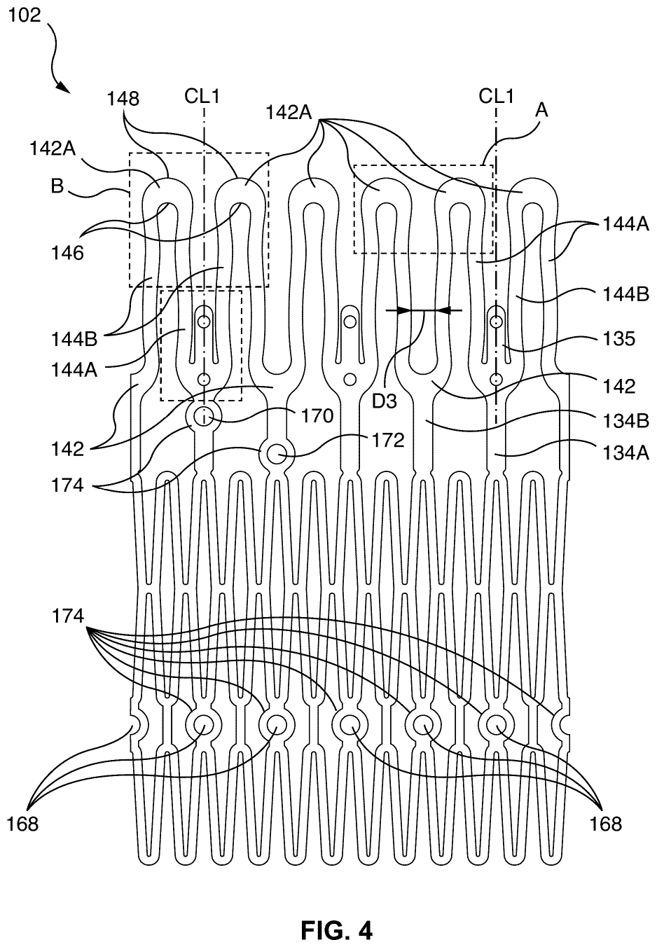

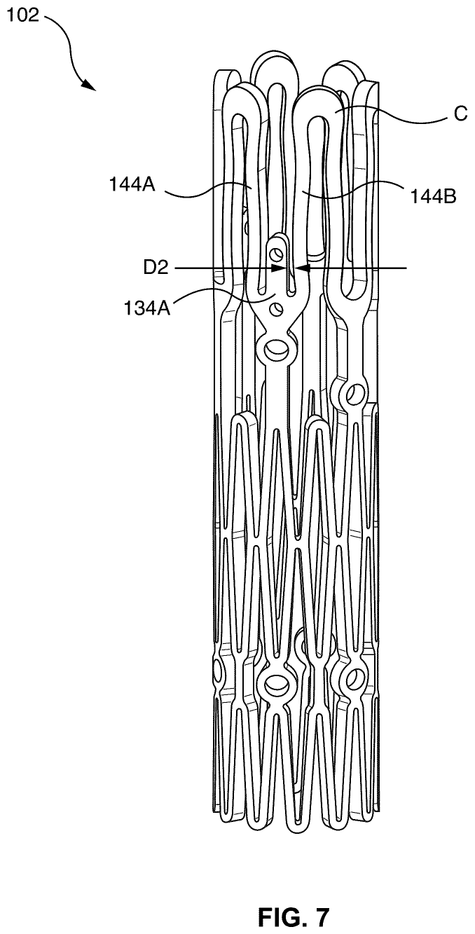

In embodiments herein, the frame 102 is a balloon expandable tubular component defining a central lumen or passageway 126, and further defining the proximal or inflow end 108 and the distal or outflow end 110 of the transcatheter heart valve prosthesis 100, as shown in FIG. 3. The frame 102 includes an expanded configuration shown in FIG. 3, an as-cut configuration shown in FIG. 4, and a non-expanded or crimped configuration shown in FIG. 7. The expanded configuration as used herein refers to the configuration of the frame 102 after expansion by a balloon catheter, other mechanical expansion, or self-expansion in a self-expanding frame. The as-cut configuration as used herein refers to the configuration of the frame 102 when the frame 102 is being formed or cut. The non-expanded or crimped configuration as used herein refers to the configuration of the frame 102 after crimping the frame 102 onto a balloon of a balloon catheter for delivery or radially compressing the frame 102 into a delivery device for delivery. When the frame 102 is in the expanded configuration of FIG. 3, an outflow diameter DO of the outflow end 110 of the transcatheter heart valve prosthesis 100 is larger than an inflow diameter D1 of the inflow end 108, as described below. In an embodiment, the inflow diameter D1 may range between 18 mm and 30 mm and the outflow diameter DO may range between 20 mm and 35 mm. The outflow diameter DO is also larger than a transition diameter DT at the transition portion 122 of the frame 102. The transition diameter DT may also be in the range between 18 mm and 30 mm. The frame 102 may be formed by a laser-cut manufacturing method and/or another conventional stent forming method as would be understood by one of ordinary skill in the art. The frame 102 is formed from a plastically deformable material such that when expanded, the frame 102 maintains its radially expanded configuration or state. The frame 102 may be formed from stainless steel or other suitable metal, such as, but not limited to platinum iridium, cobalt chromium alloys such as MP35N, platinum-chromium alloys, molybdenum rhenium, or various types of polymers or other materials known to those skilled in the art, including said materials coated with various surface deposits to improve clinical functionality.

As shown in FIG. 3, the frame 102 includes an inflow portion 120, an outflow portion 122, and a transition portion 124 bridging, connecting, or otherwise extending between the inflow portion 120 and the outflow portion 122.

The inflow portion 120 of the frame 102 may be proximate the inflow end 108 of the frame 102 and the transcatheter heart valve prosthesis 100. The inflow portion 120 includes a plurality of rows 127 formed of a plurality inflow crowns 128 and a plurality of struts 130, with each inflow crown 128 being formed between a pair of opposing struts 130. Each inflow crown 128 is a segment or bend extending between opposing struts 130. The inflow portion 120 is tubular, with a plurality of side openings or cells 132 being defined by the plurality of inflow crowns 128 and the plurality of struts 130. The inflow portion 120 further includes a plurality of junctions 129 longitudinally connecting adjacent rows 127 of the inflow portion 120. The junctions 129 may be formed by directing connecting the inflow crowns 128 of adjacent rows 127 of the inflow portion 120 or by longitudinal connectors connection the inflow crowns 128 of adjacent rows 127 of the inflow portion 120. Radiopaque markers 131 may be provided at some of the junctions 129, as shown in FIG. 3. The length or height of the inflow portion 120 may vary from that depicted herein in order to accommodate dimensions of the native valve anatomy.

The transition portion 124 bridges, connects, or otherwise extends between the inflow portion 120 and the outflow portion 122. While the stent frame has been described as including a transition portion 124, one skilled in the art will realize that the transition portion 124 may form a portion of the inflow portion 120 and/or the outflow portion 122. The transition portion 124 includes a minimum of three axial frame members 134, each axial frame member 134 extending between an outflow crown 142 of the outflow portion 122 and an inflow crown 128 of the inflow portion 120. The axial frame members 134 are substantially parallel to the central longitudinal axis of the frame 102. Each axial frame member 134 is disposed approximately halfway between a pair of adjacent endmost outflow crowns 142A. In an embodiment, the transition portion 124 includes up to six axial frame members 134, with three of the axial frame members 134 being lengthened commissure posts 134A and three of the axial frame members 134 being axial struts 134B. The lengthened commissure posts 134A are circumferentially spaced apart and aligned with and attached to a respective commissure of the three leaflets of the prosthetic valve, and the axial struts 134B are disposed between adjacent commissure posts 134A. The axial frame members 134 aid in valve alignment and coaptation. More particularly, the axial frame members 134 reinforce or strengthen the commissure region of the prosthetic valve by shaping the leaflets and supporting the leaflets during opening and closing thereof, and thus provide more reliable leaflet coaptation.

The lengthened commissure posts 134A of the frame 102 are formed to have an axial length greater than the axial struts 134B. Each lengthened commissure posts 134A extends into the outflow portion 124 of the frame 102 to allow for lengthened commissure posts without increasing the overall height of the transcatheter valve prosthesis 100. More particularly, each axial strut 134B is an axial segment having a first end connected to an outflow crown 142 of the outflow portion 124 and a second end connected to an inflow crown 128 of the inflow portion 120. Stated another way, one of the outflow crowns 142 of the outflow portion 124 may be considered the outflow end of each axial strut 134B and one of the inflow crowns 128 of the inflow portion 120 may be considered the inflow end of each axial strut 134B. Conversely, each lengthened commissure post 134A has a first end connected to one of the inflow crowns 128 of the inflow portion 120 while a second, unattached or free end of each the lengthened commissure post 134A is disposed within the outflow portion 124. Stated another way, one of the inflow crowns 128 of the inflow portion 120 may be considered the inflow end of each commissure post 134A and the outflow end of each commissure post 134A is the unattached or free end thereof. Because the lengthened commissure posts 134A are longer than the axial struts 134B, struts 144 of the outflow portion 124 intersect the lengthened commissure posts 134A at a central or mid-portion thereof. The location of the connection between the outflow struts 144 of the outflow portion 124 to the mid-portions of the lengthened commissure posts 134A is spaced a distance, in the direction of the inflow end 108, from the unattached or free end of the lengthened commissure posts 134A and is also spaced a distance in the outflow direction from the inflow end of the lengthened commissure posts 134A. Thus, an outflow portion 135 of each commissure post 134A extends distally beyond the location of the connection between the outflow struts 144 and the commissure post 134A.

The outflow portion 122 is formed proximal to the outflow end 110 of the frame 102. The outflow portion 122 includes a plurality of outflow crowns 142 and a plurality of struts 144 with each outflow crown 142 being formed between a pair of opposing struts 144. Each outflow crown 142 is a segment or bend extending between opposing struts 144. A series of endmost outflow crowns 142A are formed at the outflow end 110 of the frame 102. In embodiments, the endmost outflow crowns 142A are not connected to axial frame members 134 of the transition portion 124 but rather may be considered to be free or unattached while the remaining outflow crowns 142 of the outflow portion 122 are connected to the axial frame members 134 and disposed closer to the inflow end 108 than the endmost outflow crowns 142A. In embodiments, the frame 102 may include a single row of struts 144 and outflow crowns 142 coupled to the axial frame members 134. Thus, a row of six endmost cells 166 are formed proximate the outflow end 110 of the frame 102, with each endmost cell 166 being defined by two outflow struts 144, one of the endmost outflow crowns 142A coupled to the two outflow struts 144, one of the axial struts 134B, one of the commissure posts 134A, four of the inflow struts 130 of the third row 127C of the inflow portion 120 closest to the axial members 134, and three of the inflow crowns 128 in the third row 127C (two of which are coupled to the commissure strut 134B and the axial strut 134A, respectively). The endmost cells 166 are large such as to enable access to the coronary arteries after implantation of the transcatheter heart valve prosthesis 100.

As noted above, in embodiments herein, the outflow end 110 of the frame 102 is configured to flare radially outward when the frame 102 is in the radially expanded configuration. More particularly, a portion of the outflow portion 122 is configured to flare radially outward when the frame 102 is in the radially expanded configuration such that the outflow diameter DO of the outflow end 110 of the transcatheter heart valve prosthesis 100 is larger than the inflow diameter D1 of the inflow end 108, as shown in FIG. 3. In an embodiment, in the radially expanded configuration, the outflow diameter DO of the outflow end 110 is in the range of about 20 mm to about 35 mm and the inflow diameter D1 of the inflow end 108 is in the range of about 18 mm to about 30 mm. In embodiments, in the radially expanded configuration, the outflow diameter DO is 18%-28% larger than the inflow diameter D1.

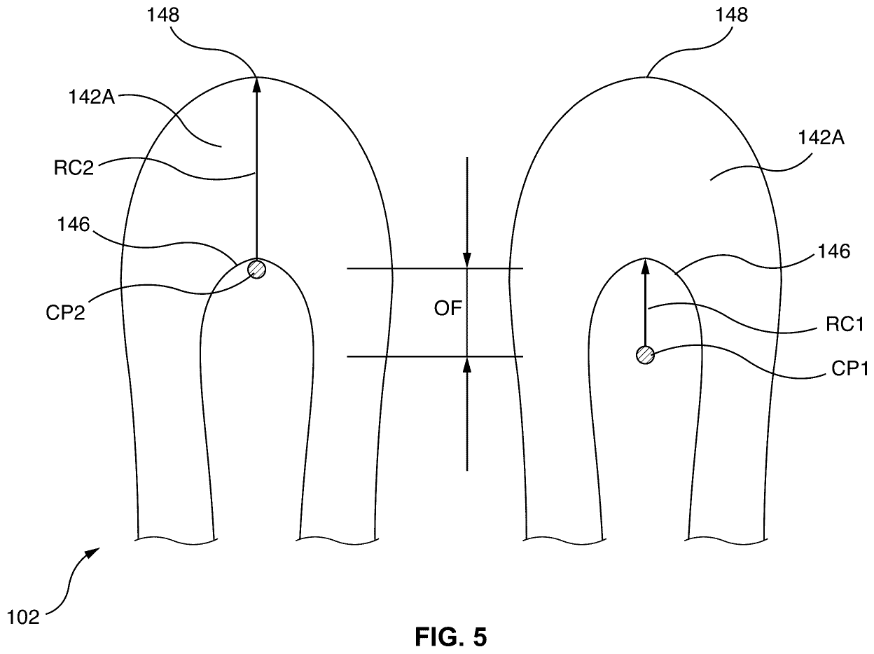

In embodiments herein, the outflow end 110 of the frame 102 is configured flare outwardly when radially expanded by having an offset OF between center point CP1 of a radius of curvature RC1 of an inner edge 146 and a center point CP2 of a radius of curvature RC2 of an outer edge 148 of the endmost outflow crowns 142A when the frame 102 is formed. In particular, the frame 102 described herein is formed by laser-cutting, etching, or otherwise removing material from a tube such that the pattern of the frame 102 remains. Thus, as shown in the laid-open as-cut configuration or state shown in FIGS. 4-5, each endmost outer crown 142A of the frame 102 includes the inner edge 146 and the outer edge 148. The center point CP1 and the radius of curvature RC1 defines a curvature of an arc of the inner edge 146, as shown in FIG. 5 which illustrates a portion of the outflow portion 122 in a laid-open view with the frame 102 in the as-cut configuration. The center point CP2 and the radius of curvature RC2 define a curvature of an arc of the outer edge 148. In embodiments, the center point CP1 (inner edge) is positioned proximal of the center point CP2 (outer edge). The offset OF of the center points CP1, CP2 of the inner edge 146 and the outer edge 148, respectively, of the endmost outflow crown 142A causes the endmost outflow crowns 142A to flare radially outward as the frame 102 transitions to the radially expanded configuration. The larger the offset OF between the center point CP1 and the center point CP2, the larger the flaring of the endmost outflow crown 142A of the outflow end 110 of the frame 102 when the frame 102 is in the radially expanded configuration. The offset OF between the center point CP1 and the center point CP2 may be selected based on the native anatomy of the valve being replaced, the desired clearance for the prosthetic valve leaflets, and/or other factors. Flaring of the outflow end 110 of the frame 102 and the transcatheter heart valve prosthesis 100 when the transcatheter heart valve prosthesis 100 is deployed within a native valve provides additional clearance between the prosthetic valve leaflets 106 and the outflow end 110 of the frame 102. This additional clearance minimizes and/or prevents the prosthetic valve leaflets 106 from contacting with the frame 102 when the prosthetic valve leaflets 106 open to permit blood flow therethrough, thereby improving valve leaflet durability, especially in native valve anatomies with extreme elliptical annuli. In embodiments herein, the flaring of the outflow end 110 of the frame 102 of the transcatheter heart valve prosthesis 100 occurs based on the offset OF between the center point CP1 of the inner edge 146 and the center point CP2 of the outer edge 148 as the frame 102 transitions to the radially expanded configuration. Therefore, the transcatheter heart valve prosthesis 100 may be deployed and the flaring will occur with a standard balloon and does not require a specially shaped balloon.

In embodiments, the radius of curvature RC1 of the inner edge 146 of each endmost outflow crown 142A may be about 0.45 mm and the radius of curvature RC2 of the outer edge 148 of each endmost outflow crown 142A may be about 1.1 mm Those skilled in the art will recognize that these are examples for a specific embodiment and a specific size frame, and that other radii of curvature may be used for different frames. In embodiments herein, the offset OF between the center point CP1 of the inner edge 146 and the center point CP2 of the outer edge 148 of each endmost outflow crown 142A may be in a range of range of 0.2 mm to 0.7 mm, or 0.3 mm to 0.6 mm, or 0.4 mm to 0.5 mm.

By configuring the outflow end 110 of the frame 102 to flare radially outwardly when radially expanded via the offset OF described above, the frame 102 may be laser cut, etched, or otherwise formed by removing material from a tube with a constant diameter. Thus, in the as-cut configuration, the outflow diameter DO_{AC} is equal to the inflow diameter DI_{AC} and to the transition diameter DT_{AC}. In the crimped configuration, the outflow diameter DO_{CR} is equal to the inflow diameter DI_{CR} and to the transition diameter DT_{CR}.

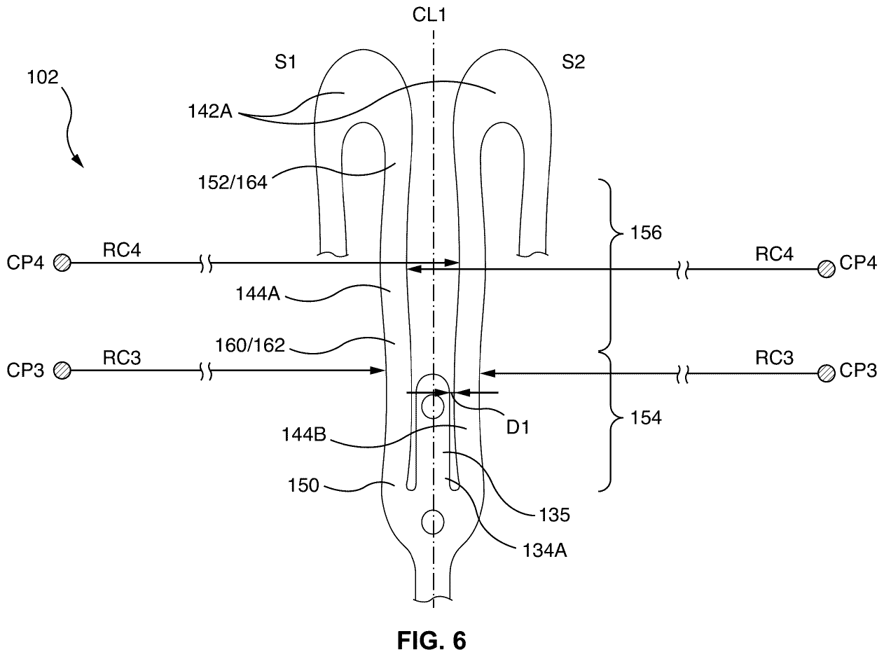

As noted above, it is also desirable to reduce or eliminate pinching of the prosthetic valve leaflets 106 between portions of the frame 102, such as the struts, the crowns, and the axial members. In particular, potential pinching of the prosthetic valve leaflets 106 may occur in the outflow portion 124 of the frame 102, such as between the outflow portions 135 of the lengthened commissure posts 134A and the outflow struts 144 of the outflow portions 124 of the frame 124. As described above, and as shown FIG. 4 and in the enlarged view of FIG. 6, the two outflow struts 144 adjacent the commissure posts 134A intersect with a mid-portion of the commissure posts 134A and extend generally in a distal direction to a corresponding endmost outflow crown 142A. Thus, at each commissure post 134A, a first outflow strut 144A is disposed on a first side S1 of a centerline CL1 of the commissure post 134A and a second outflow strut 144B is disposed on a second side S2 of the centerline CL1 of the commissure post 134A, opposite the first outflow strut 144A. In embodiments, the first strut 144A and the second strut 144B are each a mirror images of the other. The first outflow strut 144A extends generally in a distal direction to the corresponding endmost outflow crown 142A on the first side S1 of the centerline CL1 of the commissure post 134A, and the second outflow strut 144B extends generally in a distal direction to a corresponding endmost outflow crown 142A on the second side S2 of the centerline CL1 of the commissure post 134A. Although described with respect to the outflow struts 144 adjacent the commissure posts 134A, the outflow struts 144 extending from the proximal outflow crowns 142 at the axial struts 134B.

As shown in FIG. 6, each outflow strut 144 of the outflow portion 124 includes a first end 150 and a second end 152. The first end 150 extends distally from a corresponding outflow crown 142 or a lengthened commissure 134A and extends to a corresponding endmost outflow crown 142A. In embodiments herein, each outflow strut 144 is formed (i.e., in the as-cut configuration, e.g., laser-cut, etched, etc.) with a double curve creating an s-shape, shown in FIGS. 4-6. Accordingly, in the as-cut configuration of the frame 102, each strut 144 includes a first segment 154 and a second segment 156. The first segment 154 includes the first end 150 of the strut 144 and is adjacent to the corresponding proximal outflow crown 142 or lengthened commissure post 134A and a second end 160 distal of the first end 150. The first segment 154 of the strut 144 extends generally in a distal direction and curves gently circumferentially towards the centerline CL1. In other words, as shown in FIGS. 4 and 6, the first segment 154 of each outflow strut 144 forms a convex curve relative to the centerline CL1 of the corresponding proximal outflow crown 142 or commissure post 134A. The first segment 154 has a radius of curvature RC3 with a center point CP3 on a side of the first segment 154 opposite the centerline CL1 (i.e., opposite the outflow portion 135 of the commissure post 134A or opposite the proximal outflow crown 142). In embodiments, the radius of curvature RC3 of the first segment 154 may be in a range of about 10.5 mm to about 16 mm. In a non-limiting example, the radius of curvature RC3 of the first segment is about 15 mm.

The second segment 156 of each outflow strut 144 extends in a gentle curve and generally in a distal direction from a first end 162 adjacent to the second end 160 of the first segment 154 to the second end 152 of the outflow strut 144 adjacent to the corresponding endmost outflow crown 142A. In embodiments herein, the second segment 156 curves in a direction opposite the first segment 154. Thus, as shown in FIG. 6, the second segment 156 is a concave curve relative to the centerline CL1 of the corresponding commissure post 134A or the corresponding outflow crown 142. The second segment 156 has a radius of curvature RC4 with a center point CP4 on an opposite side of the centerline CL1 of the corresponding outflow crown 142 as the outflow strut 144. More particularly, for example, the first outflow strut 144A is disposed on the first side S1 of the centerline CL1. The center point CP4 of the curve of the second segment 156 of the first outflow struts 144A is on the second side S2 of the centerline CL1. Similarly, the second outflow strut 144B is disposed on the second side S2 of the centerline CL1. The center point CP4 of the curve of the second segment 156 of the second outflow strut 144B is located on the first side S1 of the centerline CL1. In embodiments, the radius of curvature RC2 of the second segment 156 may be in a range of about 9.7 mm to about 11 mm. In a non-limiting example, the radius of curvature RC2 of the second segment 156 is about 10.8 mm.

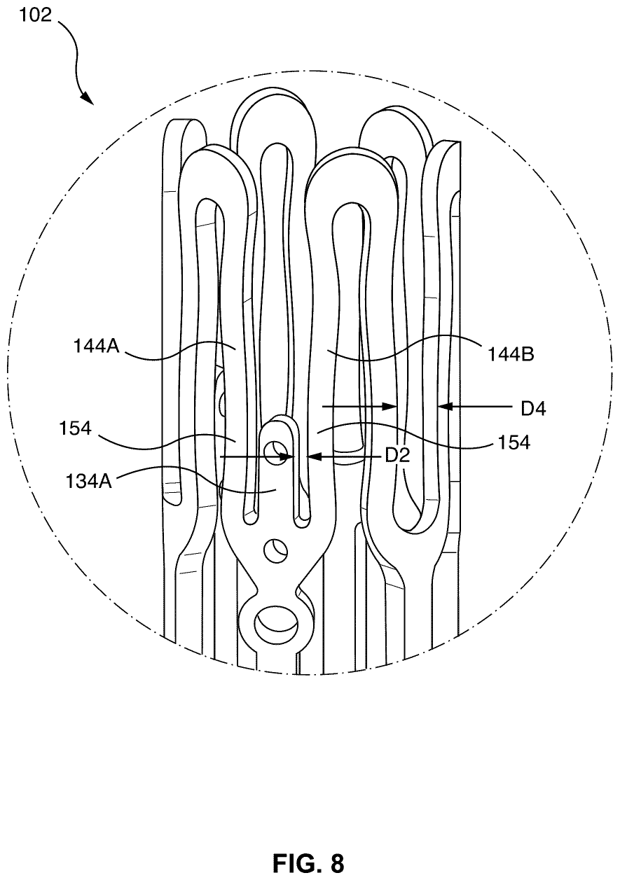

In embodiments herein, the curved shape of each strut 144 is configured to increase the distance between the first segment 154 of the strut 144 and the outflow portion 135 of the adjacent commissure post 134A when the frame 102 is compressed to the crimped configuration. More particularly, after the frame 102 is formed, the frame 102 is radially expanded from the as-cut configuration to the radially expanded configuration. After forming the heart valve prosthesis 100 (e.g., attaching the valve leaflets, skirts, etc.), the heart valve prosthesis 100 is radially compressed or crimped onto a balloon. When the frame 102 transitions to the crimped configuration, each outflow strut 144 transitions from the s-curve shape in the as-cut configuration to a more linear shape that increases the distance between the outflow strut 144 and the outflow portion 135 of an adjacent commissure post 134A. As shown in FIG. 6, when the frame 102 is in the as-cut configuration, a first distance D1 is defined between the first segment 154 of each outflow strut 144 and its corresponding adjacent outflow portion 135 of the adjacent commissure post 134A. After the frame 102 is radially expanded and then crimped to the crimped configuration, a second distance D2 is defined between the first segment 154 of each outflow strut 144 and its corresponding outflow portion 135 of the adjacent commissure post 134A, as shown in FIGS. 7-8. In embodiments herein, the second distance D2 is larger than the first distance D1. The larger or increased second distance D2 between each outflow strut 144 and the adjacent outflow portion 135 of the commissure post 134A minimizes or prevents pinching or binding of the leaflets 106 of the valve component 104 between the outflow struts 144 and the outflow portions 135 of the commissure posts 134A when the transcatheter heart valve prosthesis 100 is crimped onto a balloon catheter, thereby preventing leaflet damage and improving valve leaflet durability. In embodiments herein, the first distance D1 may be in the range of 0.06 mm to 0.10 mm, or about 0.08 mm and the second distance D2 may be in the range of 0.50 mm to 0.60 mm.

In the area of the axial struts 134B, the re-shaping of the outflow struts 144 from the as-cut configuration to the radially compressed or crimped configuration, as described above, results in the first segments 154 of adjacent outflow struts 144 coupled to a common outflow crown 142 being spaced farther apart from each other in the radially compressed or crimped configuration than in the as-cut configuration. In other words, the first segments 154 of outflow struts 144 coupled to a common axial strut 134B (i.e., not the location of a commissure post 134A) are spaced a third distance D3 from each other in the as-cut configuration, as shown in FIG. 4, and are spaced a fourth distance D4 from each other in the radially compressed configuration, as shown in FIG. 8. The fourth distance D4 is greater than the third distance D3. The larger fourth distance D4 between the first segments 154 of the outflow struts 144 minimizes or prevents pinching or binding of the leaflets 106 of the valve component 104 between the outflow struts 144 when the transcatheter heart valve prosthesis 100 is crimped onto a balloon catheter, thereby preventing leaflet damage and improving valve leaflet durability. In embodiments herein, the third distance D3 may be in the range of about 0.48 mm to about 0.88 mm, or about 0.68 mm and the fourth distance D4 may be in the range of about 0.80 mm to about 1.20 mm, or about 1.0 mm.

It is noted that the first distance D1, the second distance D2, the third distance D3, and the fourth distance D4 are measured circumferentially or laterally.

While the frame 102 is described herein including a single row of struts 144 and outflow crowns 142, this is not meant to be limiting. One skilled in the art will realize that the above configuration of the outflow portion 122 may include additional rows of struts and crowns.

In embodiments, to ensure the proper placement in the native anatomy of a subject, the frame 102 may include one or more inflow markers 168 positioned in the inflow portion 120, and a first outflow marker 170 and a second outflow marker 172 positioned in the transition portion 124 of the frame 102, as shown in FIG. 4. The inflow markers 168, the first outflow marker 170, and the second outflow marker 172 may be utilized to orient the transcatheter heart valve prosthesis 100 in orientation (e.g., axial/annular alignment, tilt alignment, circumferential (rotational) alignment, etc.), in situ, as would be understood by one of ordinary skill in the art.

In embodiments, the inflow markers 168, the first outflow marker 170, and the second outflow marker 172 may each be attached to the frame 102 within a containment member 174. The containment member 174 may be a hollow structure or opening in the frame 102 which is configured to receive a corresponding inflow marker 168, first outflow marker 170, or second outflow marker 172. Accordingly, each containment member 174 is configured in a shape that matches a shape of the corresponding marker.

While FIGS. 1-8 illustrate one example of the positioning and number of inflow markers 168 within the inflow portion 120, one skilled in the art will realize that the frame 102 may include any number of inflow and/or outflow markers positioned at any location in the frame 102. In particular, the transcatheter heart valve prosthesis 100 may include within the inflow and/or outflow markers as shown and described in U.S. Pat. No. 12,064,343 to Medtronic, Inc., U.S. Patent Application Publication No. 2022/0061985 to Medtronic, Inc., and International Publication No. WO 2023/031829 to Medtronic, Inc., each of which is incorporated by reference herein in its entirety. While embodiments of the frame 102 of FIGS. 1-8 have been described with one or more inflow markers 168 positioned in the inflow portion 120, and a first outflow marker 170 and a second outflow marker 172 positioned in the transition portion 124 of the frame 102, this is not meant to be limiting. In other embodiments, the one or more inflow markers, the first outflow marker, and the second outflow marker may be omitted from the frame to reduce the crimped crossing profile of the transcatheter heart valve prosthesis.

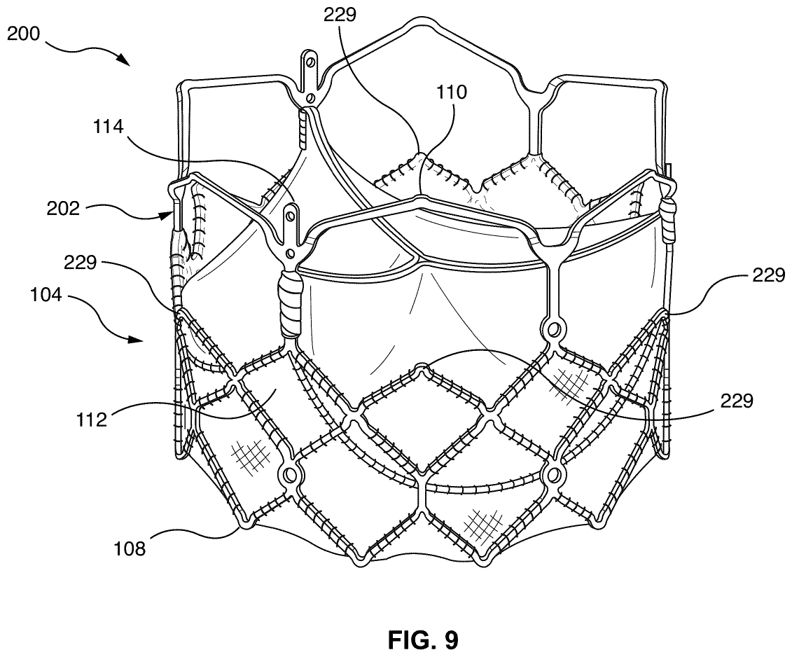

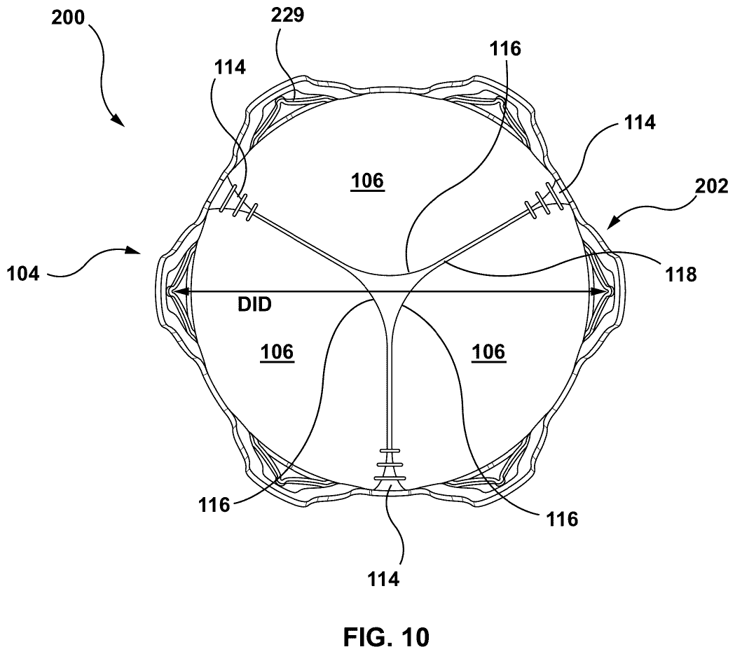

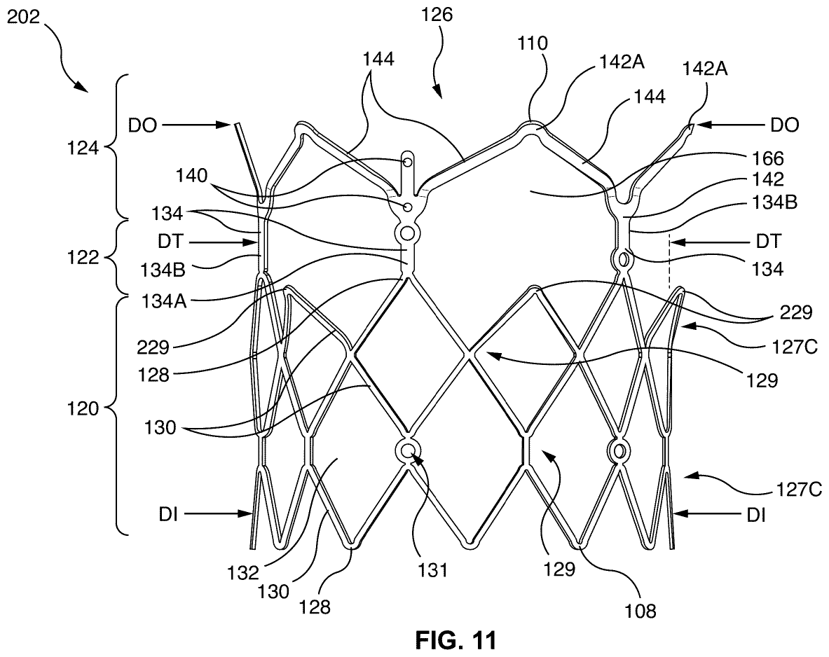

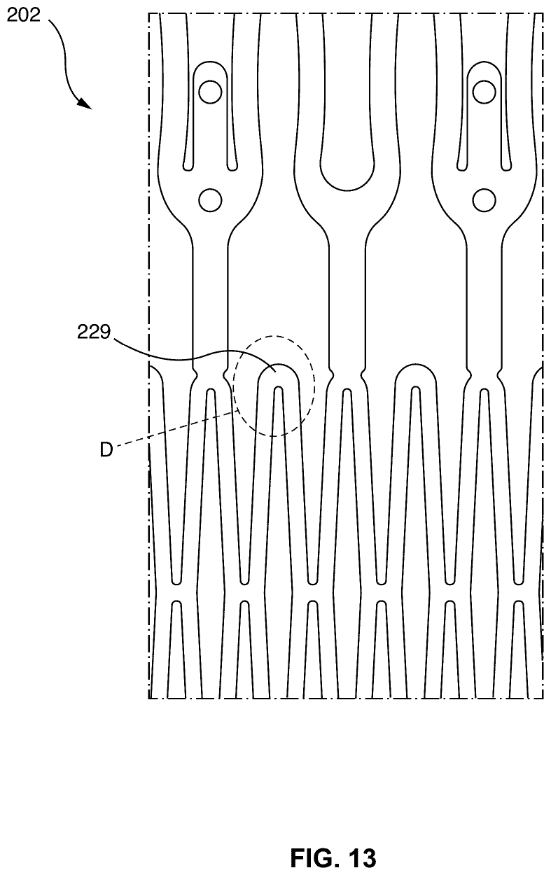

FIGS. 9-14B show a transcatheter heart valve prosthesis 200 according to another embodiment hereof. The transcatheter heart valve prosthesis 200 is the same as the transcatheter heart valve prosthesis of FIGS. 1-8 except as explained herein. Therefore, the same reference numbers are used for the transcatheter heart valve prosthesis 200 as used for the transcatheter heart valve prosthesis 100 except for the differences. In particular, the transcatheter heart valve prosthesis 200 includes a radially-expandable frame 202 and a prosthetic valve component 104. The prosthetic valve component 104 is the same as the prosthetic valve component 104 described above. Therefore, the description thereof will not be repeated and the description above, with all variations, is incorporated into the description of the transcatheter heart valve prosthesis 200. The frame 202 is generally the same as the frame 102 except for certain crowns at the outflow/distal end of the inflow portion 120 of the frame 202. Therefore, the details of the frame 102 described above are incorporated into the description of the frame 202 except for the changes described below.

In particular, as described above, the frame 202 generally includes an inflow portion 120, an outflow portion 122, and a transition portion 124 bridging, connecting, or otherwise extending between the inflow portion 120 and the outflow portion 122. The description of the portions above is incorporated into the description of the frame 202. The frame 202 differs in that some of the inflow crowns 128 of the frame 202 differ from the inflow crowns 128 of the frame 102. In particular, in the frame 202, distal inflow crowns 229 that are not coupled to an axial frame member 134, which also may be referred to as free or unattached distal inflow crowns 229, are configured to flare radially outwardly when the frame 202 is in the radially expanded configuration. More particularly, the distal inflow crowns 229 are configured to flare radially outwardly when the frame 202 is in the radially expanded configuration such that a distal inflow portion diameter D_{ID} at the free distal inflow crowns 229 is larger than transition portion diameter DT at the axial frame members 134. In an embodiment, the distal inflow transition portion diameter D_{ID} at the free distal inflow crowns 229 is in the range of about 18.4 mm to about 30.6 mm while the transition portion diameter DT is in the range of about 18 mm to about 30 mm. In other words, each free distal inflow crown 229 flares outwardly about 0.20 mm to about 0.30, or about 0.20 mm to about 0.25 mm. In embodiments, in the radially expanded configuration, the distal inflow portion diameter D_{ID} is about 1% to about 4% larger than the transition portion diameter DT at the axial frame members 134.

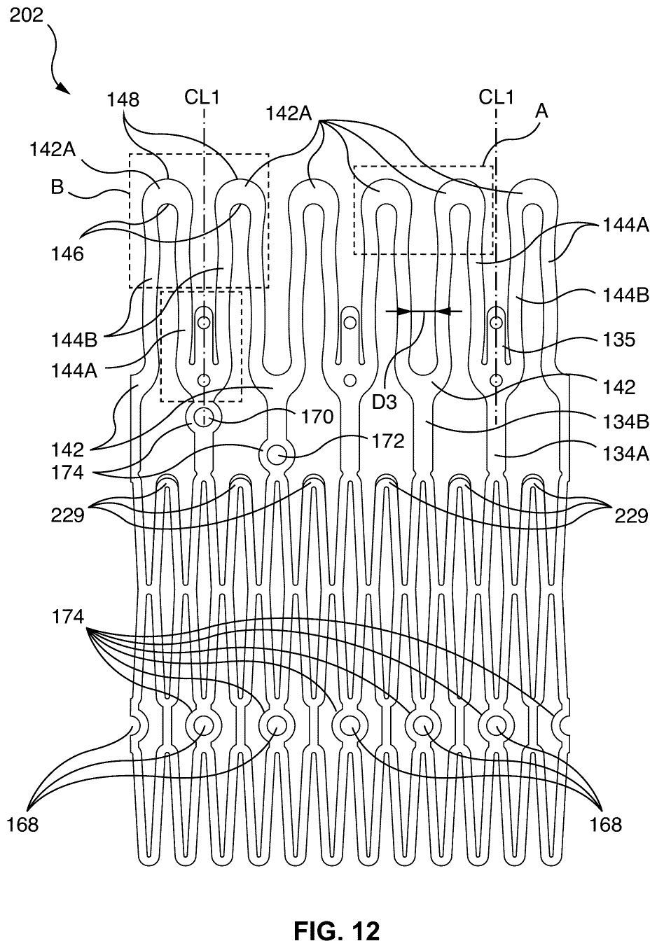

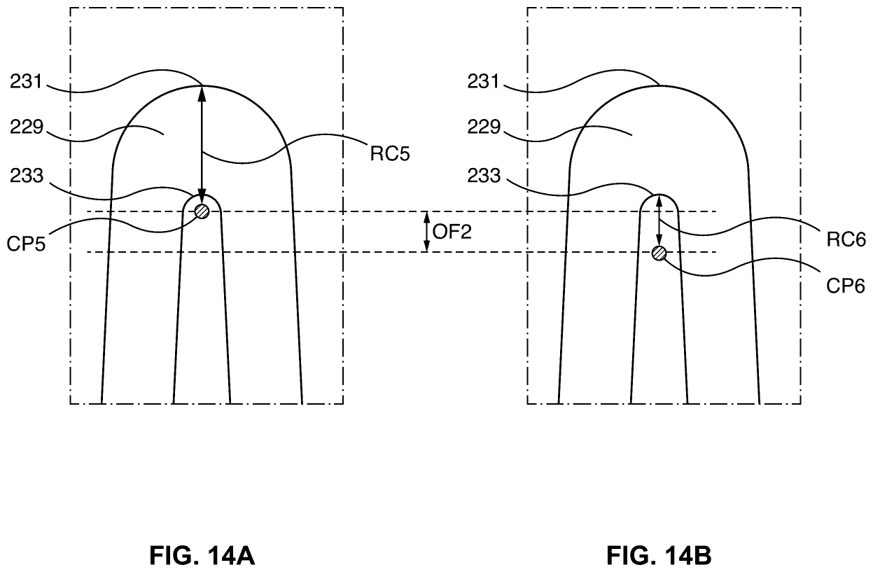

In embodiments herein, the free distal inflow crowns 229 of the frame 202 are configured flare outwardly when radially expanded by having an offset OF between a center point CP5 of a radius of curvature RC5 of an outer edge 231 and a center point CP6 of a radius of curvature RC6 of an inner edge 233 of the free distal inflow crowns 229 when the frame 202 is formed. In particular, the frame 202 described herein is formed by laser-cutting, etching, or otherwise removing material from a tube such that the pattern of the frame 202 remains. Thus, as shown in the laid-open as-cut configuration or state shown in FIGS. 12-14B, each free distal inflow crown 229 of the frame 202 includes the inner edge 233 and the outer edge 231. The center point CP5 and the radius of curvature RC5 defines a curvature of an arc of the outer edge 231, as shown in FIG. 14A which illustrates a one of the free distal inflow crowns 229 in a laid-open view with the frame 202 in the as-cut configuration. The center point CP6 and the radius of curvature RC6 defines a curvature of an arc of the inner edge 233, as shown in FIG. 14B which illustrates a one of the free distal inflow crowns 229 in a laid-open view with the frame 202 in the as-cut configuration. In embodiments, the center point CP6 (inner edge) is positioned proximal of the center point CP5 (outer edge). The offset OF2 of the center points CP5, CP6 of the inner edge 233 and the outer edge 231, respectively, of the free distal inflow crowns 229 causes the free distal inflow crowns 229 to flare radially outward as the frame 202 transitions to the radially expanded configuration. The larger the offset OF2 between the center point CP6 and the center point CP5, the larger the flaring of the free distal inflow crowns 229 at the distal end of the inflow portion 120 when the frame 202 is in the radially expanded configuration. The offset OF2 between the center point CP6 and the center point CP5 may be selected based on the native anatomy of the valve being replaced, the desired clearance for the prosthetic valve leaflets, and/or other factors. Flaring of the free distal inflow crowns 229 at the distal end of the inflow portion 120 of the frame 202 when the transcatheter heart valve prosthesis 200 is deployed within a native valve provides additional clearance between the prosthetic valve leaflets 106 and distal end of the inflow portion 120 of the frame 202. This additional clearance minimizes and/or prevents the prosthetic valve leaflets 106 from contacting with the frame 202 when the prosthetic valve leaflets 106 open to permit blood flow therethrough, thereby improving valve leaflet durability, especially in native valve anatomies with extreme elliptical annuli. In embodiments herein, the flaring of the free distal inflow crowns 229 at the distal end of the inflow portion 120 of the frame 202 of the transcatheter heart valve prosthesis 200 occurs based on the offset OF between the center point CP6 of the inner edge 233 and the center point CP5 of the outer edge 231 as the frame 202 transitions to the radially expanded configuration. Therefore, the transcatheter heart valve prosthesis 200 may be deployed and the flaring will occur with a standard balloon and does not require a specially shaped balloon.

In embodiments, the radius of curvature RC6 of the inner edge 233 of each free distal inflow crown 229 may be about 0.09 mm and the radius of curvature RC5 of the outer edge 231 of each free distal inflow crown 229 may be about 0.45 mm. Those skilled in the art will recognize that these are examples for a specific embodiment and a specific size frame, and that other radii of curvature may be used for different frames. In embodiments herein, the offset OF2 between the center point CP6 of the inner edge 233 and the center point CP5 of the outer edge 231 of each free distal inflow crown 229 may be in a range of range of about 0.2 mm to about 0.5 mm, or about 0.3 mm to about 0.4 mm. In the embodiment shown the offset OF2 is about 0.2 mm.

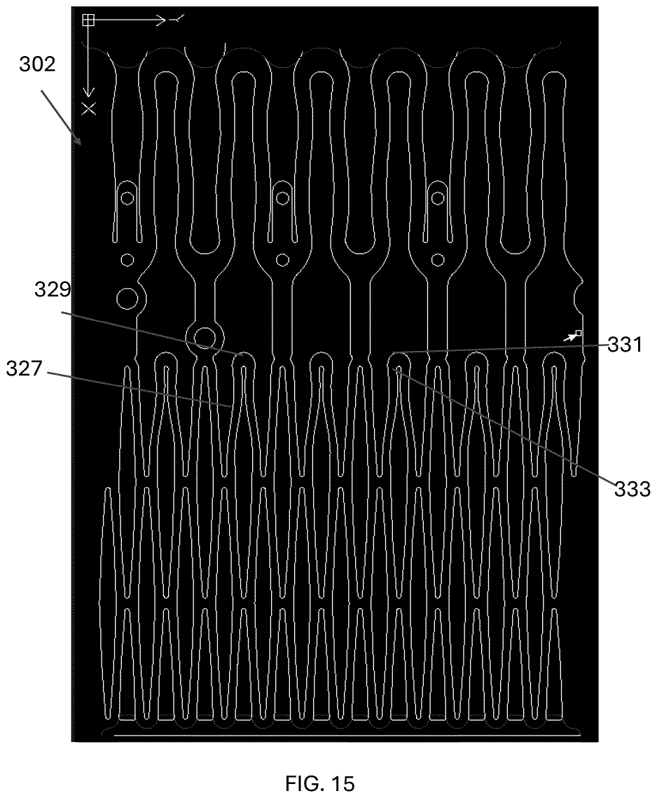

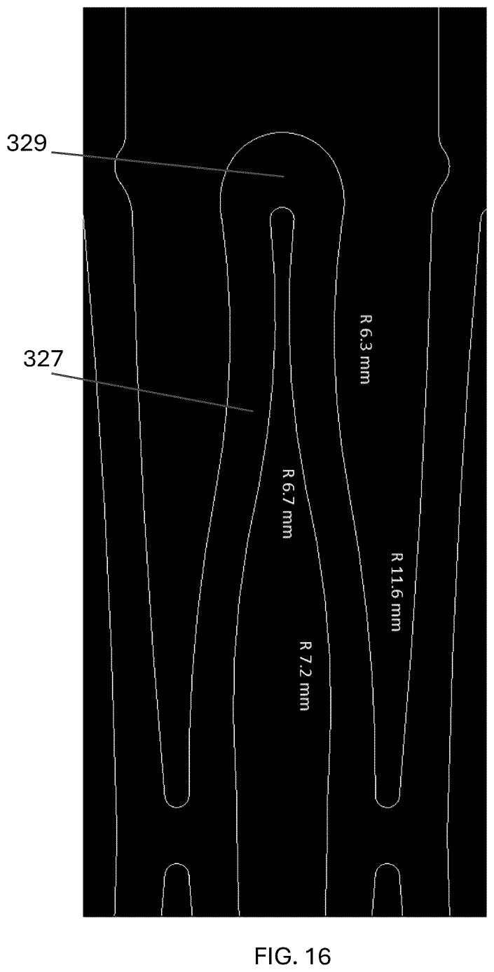

FIGS. 15-16 show a frame 302 a transcatheter heart valve prosthesis according to another embodiment hereof. FIG. 15 shows the frame 302 in a flattened or laid-open, as-cut view. The frame 302 can be used with a transcatheter heart valve prosthesis such as the transcatheter heart valve prosthesis shown in FIG. 1 or FIG. 9. Thus, the heart valve prosthesis include a prosthetic heart valve including leaflets (not shown) as described above. The frame 302 is the same as the frame 202 described above except for the differences described herein.

In particular, the outflow portion 122 of the frame 302 is as described above with respect to FIGS. 1-8. Further, the inflow portion 120 of the frame 302 includes distal inflow crowns 329 that flare radially outwardly, as described with respect to FIGS. 9-14B. However, the frame 302 further includes S-shaped struts 327 of the inflow portion 120 that are coupled to the distal inflow crowns 329. Thus, as can be seen in FIG. 15, the struts 331 coupled to the distal inflow crowns 329 and extend proximally therefrom (i.e., towards the inflow end of the frame 302). The struts 327 are S-shaped, as shown in FIG. 15, and similar to as described above with respect to the struts 144A, 114B of the outflow portion 122. FIG. 16 shows a close-up view of the S-shaped struts 327, showing the radii of curvature for the different portions of each S-shape strut 327.

Further, the distal inflow crowns 329 of the embodiment of FIG. 15 are similar to the the distal inflow crows 229 of FIGS. 9-14B in that the distal inflow crowns 329 have radii of curvature for the inner edge 333 and the outer edge 331 of the distal inflow crowns 329 that result in an offset between the centerpoints thereof. In particular, in the embodiment of FIG. 15, similar to the embodiment of FIGS. 9-14B, the radius of curvature of the outer edge 331 may be about 0.09 mm and the radius of curvature of the inner edge 333 may be about 0.45 mm, with an offset between the centerpoint of the radius of curvature of the inner edge 333 and the centerpoint of the radius of curvature or the outer edge 331 of about 0.2 mm.

While various embodiments have been described above, it should be understood that they have been presented only as illustrations and examples of the present technology, and not by way of limitation. It will be apparent to persons skilled in the relevant art that various changes in form and detail may be made therein without departing from the spirit and scope of the present technology. Thus, the breadth and scope of the present technology should not be limited by any of the above-described embodiments but should be defined only in accordance with the appended claims and their equivalents. It will also be understood that each feature of each embodiment discussed herein, and of each reference cited herein, may be used in combination with the features of any other embodiment. All patents and publications discussed herein are incorporated by reference herein in their entirety.

What is claimed is:

1. A transcatheter valve prosthesis comprising:
a frame formed in an as-cut configuration, being radially expandable to a radially expanded configuration and radially compressible to a crimped configuration for delivery within a vasculature, the frame including:
an inflow portion formed proximate to an inflow end of the frame, the inflow portion including a plurality or rows of inflow struts and inflow crowns, with each inflow crown being formed between a pair of opposing inflow struts,
an outflow portion formed proximate to an outflow end of the frame, the outflow portion including a plurality of endmost outflow crowns and a plurality of outflow struts with each endmost outflow crown being formed between a pair of opposing outflow struts, and
a transition portion extending between the inflow portion and the outflow portion, the transition portion including a plurality of axial frame members, the plurality of axial frame members including a plurality of commissure posts, and
a prosthetic valve disposed within and secured to at least the transition portion of the frame, the prosthetic valve being configured to substantially block blood flow in one direction to regulate blood flow through a central lumen of the frame;
wherein in the radially expanded configuration, the outflow end of the frame flares radially outwardly such that the outflow end has a first diameter larger than a second diameter at the inflow end of the frame;
wherein each endmost outflow crown has an inner edge and an outer edge, and wherein in the as-cut configuration a first center point of a first radius of curvature of the inner edge is offset from a second center point of a second radius of curvature of the outer edge such that the endmost outflow crown flares radially outward when the frame is radially expanded.

2. The transcatheter valve prosthesis of claim 1, wherein the first center point of the first radius of curvature is proximal to the second center point of the second radius of curvature.

3. The transcatheter valve prosthesis of claim 1, wherein an offset distance between the first center point and the second center point is between about 0.2 mm and about 0.7 mm.

4. The transcatheter valve prosthesis of claim 1, wherein the first radius of curvature is about 0.45 mm and the second radius of curvature is about 1.1 mm.

5. The transcatheter valve prosthesis of claim 1, wherein each outflow strut of the outflow portion has an s-shape with the frame in the as-cut configuration, and each strut of the outflow portion has a generally linear shape with the frame in the crimped configuration.

6. The transcatheter valve prosthesis of claim 5, wherein each outflow strut includes a first segment extending from a proximal end of the outflow strut in a distal direction to a second segment extending from the first segment in a distal direction to a corresponding endmost outflow crown.

7. The transcatheter valve prosthesis of claim 6, wherein the first segment of each outflow strut has a convex shape relative to a centerline of an adjacent axial member of the plurality of axial members with the frame in the as-cut configuration, and wherein the second segment of each outflow strut has a convex shape relative to the centerline of the adjacent axial member with the frame in the as-cut configuration.

8. The transcatheter valve prosthesis of claim 7,
wherein each commissure post includes an outflow portion such that outflow struts adjacent to each commissure post intersect the commissure post proximal of a free end of the commissure post,
wherein in the as-cut configuration, a first circumferential distance is defined between the first segment of a first outflow strut of the plurality of the outflow struts adjacent the commissure post and the commissure post,
wherein in the crimped configuration, a second circumferential distance is defined between the first segment of the first outflow strut and the commissure post, and
wherein the second circumferential distance is larger than the first circumferential distance.

9. The transcatheter heart valve prosthesis of claim 1, wherein free distal inflow crowns of the inflow crowns at a distal end of the inflow portion of the frame flare radially outwardly with the transcatheter heart valve prosthesis in the radially expanded configuration.

10. The transcatheter heart valve prosthesis of claim 9, wherein each of the free distal inflow crowns has an inner edge and an outer edge, and wherein in the as-cut configuration a third center point of a third radius of curvature of the inner edge is offset from a fourth center point of a fourth radius of curvature of the outer edge such that the free distal inflow crown flares radially outward when the frame is radially expanded.

11. The transcatheter heart valve prosthesis of claim 10, wherein the fourth center point is offset from the third center point by about 0.2 mm to about 0.5 mm.

12. A transcatheter valve prosthesis comprising:
a frame formed in an as-cut configuration, being radially expandable to a radially expanded configuration and radially compressible to a crimped configuration for delivery within a vasculature, the frame including:
an inflow portion formed proximate to an inflow end of the frame, the inflow portion including a plurality or rows of inflow struts and inflow crowns, with each inflow crown being formed between a pair of opposing inflow struts,
an outflow portion formed proximate to an outflow end of the frame, the outflow portion including a plurality of outflow crowns and a plurality of outflow struts with each outflow crown being formed between a pair of opposing outflow struts, and
a transition portion extending between the inflow portion and the outflow portion, the transition portion including a plurality of axial frame members, the plurality of axial frame members including a plurality of commissure posts, and
a prosthetic valve disposed within and secured to at least the transition portion of the frame, the prosthetic valve being configured to substantially block blood flow in one direction to regulate blood flow through a central lumen of the frame;
wherein each outflow strut of the outflow portion has an s-shape with the frame in the as-cut configuration, and each strut of the outflow portion has a generally linear shape with the frame in the crimped configuration.

13. The transcatheter valve prosthesis of claim 12, wherein each outflow strut includes a first segment extending from a proximal end of the outflow strut in a distal direction to a second segment extending from the first segment in a distal direction to a corresponding endmost outflow crown.

14. The transcatheter valve prosthesis of claim 13, wherein the first segment of each outflow strut has a convex shape relative to a centerline of an adjacent axial member of the plurality of axial members with the frame in the as-cut configuration, and wherein the second segment of each outflow strut has a convex shape relative to the centerline of the adjacent axial member with the frame in the as-cut configuration.

15. The transcatheter valve prosthesis of claim 14,
wherein each commissure post includes an outflow portion such that outflow struts adjacent to each commissure post intersect the commissure post proximal of a free end of the commissure post,
wherein in the as-cut configuration, a first circumferential distance is defined between the first segment of a first outflow strut of the plurality of the outflow struts adjacent the commissure post and the commissure post,
wherein in the crimped configuration, a second circumferential distance is defined between the first segment of the first outflow strut and the commissure post, and
wherein the second circumferential distance is larger than the first circumferential distance.

16. The transcatheter valve prosthesis of claim 12, wherein the outflow portion includes a plurality of endmost outflow crowns formed at the outflow end of the frame, wherein each endmost outflow crown has an inner edge and an outer edge, and wherein in the as-cut configuration a first center point of a first radius of curvature of the inner edge is offset from a second center point of a second radius of curvature of the outer edge such that the endmost outflow crown flares radially outward when the frame is radially expanded.

17. The transcatheter valve prosthesis of claim 16, wherein the first center point of the first radius of curvature is proximal to the second center point of the second radius of curvature.

18. The transcatheter valve prosthesis of claim 16, wherein the first radius of curvature is about 0.45 mm and the second radius of curvature is about 1.1 mm.

19. The transcatheter heart valve prosthesis of claim 16, wherein free distal inflow crowns of the inflow crowns at a distal end of the inflow portion of the frame flare radially outwardly with the transcatheter heart valve prosthesis in the radially expanded configuration.

20. The transcatheter heart valve prosthesis of claim 19, wherein each of the free distal inflow crowns has an inner edge and an outer edge, and wherein in the as-cut configuration a third center point of a third radius of curvature of the inner edge is offset from a fourth center point of a fourth radius of curvature of the outer edge such that the free distal inflow crown flares radially outward when the frame is radially expanded.
